(19) United States
(12) Patent Application Publication
(10) Pub. No.: US 2020/0330124 A1
(43) Pub. Date: Oct. 22, 2020

(54) GUIDING DEVICE FOR INSERTING AND GUIDING A HARVESTING FAT TISSUE CANNULA AND RELATED STERILE KIT FOR HARVESTING ADIPOSE DERIVED STEM CELLS (ADSCS)

(71) Applicant: Alessandro Gennai, Bologna (BO) (IT)

(72) Inventor: Alessandro Gennai, Bologna (BO) (IT)

(21) Appl. No.: 16/762,582

(22) PCT Filed: Nov. 9, 2018
(86) PCT No.: PCT/IB2018/058823

§ 371 Date: May 8, 2020

(30) Foreign Application Priority Data
Nov. 10, 2017 (IT) 102017000128569

(51) Int. Cl. A61B 17/34; A61B 10/02

(52) U.S. Cl. CPC A61B 17/3403; A61B 17/3421; A61B 2017/3409; A61B 2017/3407; A61B 10/0283

(57) ABSTRACT

This disclosure provides a guiding device shaped so as to hold a syringe equipped with a harvesting cannula and to allow to insert the harvesting cannula at the exact depth below the skin of the patient to attain the subcutaneous adipose tissue. The cannula may be moved move back and forth in a driven manner immersed in the subcutaneous adipose tissue without any risk of wounding inner organs of the patient and in the proper layer. This disclosure provides also a syringe holder in the form of a hollow box with circular openings on a top surface. The circular openings are shaped so as to let a barrel of a syringe enter therein and a barrel flange not pass therethrough, in order to keep harvesting syringes hung with their barrel flanges abutted against the top surface of the syringe holder and their barrels protected into the hollow box.

### TECHNICAL FIELD

This disclosure relates to devices for harvesting adipose derived stem cells (ADSCs) and more in particular to a guiding device for inserting and guiding a harvesting tissue cannula in the subcutaneous adipose tissue of a patient, and to a related sterile kit to obtain adipose derived stem cells from the subcutaneous adipose tissue.

### BACKGROUND

ADSCs are mesenchymal stem cells characterized by the ability to renew themselves through mitotic cell division and differentiate into a diverse range of specialized cell types. The ADSCs (Adipose Derived Stem Cells) are cells that abundantly exist in the SVF (Stromal Vascular Fraction) of the adipose tissue, and therefore, they are ideal as a cell source in the field of regenerative medicine. They may be obtained from fat depots, and are harvested from adipose tissue through a simple, minimally invasive minimal-invasive harvesting procedure performed by a surgeon under general or local anesthesia. Harvesting procedure may be carried out for example according to the so-called The SEFFI and MicroSEFFI (or M-SEFFI) techniques, disclosed in the article “Superficial enhanced fluid fat injection (SEFFI and MicroSEFFI) in facial rejuvenation”, by A. Gennai and F. P. Bernardini, CellR4 2017; 5 (1): e2239, and in the article “Skin Rejuvenation and Volume Enhancement with the Micro Superficial Enhanced Fluid Fat Injection (M-SEFFI) for Skin Aging of the Periocular and Perioral Regions”, by A. Gennai et al. According to these techniques, the harvesting procedure is performed using especially designed harvesting cannulas, that are depicted in FIG. 1. In order to get adipose tissue, these special cannulas are inserted under the skin and into the fat and are moved back and forth to collect adipose tissue in a syringe. The extraction of the mesenchymal stem cell ADSCs is usually done under local anaesthesia and may involve the following process steps:
1. previous local anaesthesia, through a little incision in the skin, inserting the cannula in the subcutaneous adipose tissue.
2. Harvesting procedure is performed collecting subcutaneous adipose tissue using the special harvesting cannula; the cannula is connected to a syringe. The adipose tissue with SVF and ADSCs is collecting in the syringe by creating a manual depression inside the syringe. The small side port holes cannula permit to collect small clusters of adipose tissue containing adipocytes, SVF and ADSCs.
3. After harvesting in the syringe a suitable compound of adipose tissue (an average of about half the syringe capacity), removing the cannula from the syringe, filling the syringe with Ringer lactate and closing the syringe with a cap.
4. Maintaining the syringe in the vertical position and in cold Ringer lactate in order to guarantee the cells viability. After few minutes there is the separation of tissue from washing liquid.
5. Removing washing liquid and leaving only the tissue with adipocytes, SVF with ADSCs in the syringe.
6. Proceeding with a gentle centrifuge (optional).
7. At the end of the centrifuge, removing oil and washing liquid again and leaving only tissue in the syringes containing adipocytes, SVF with ADSCs.
8. Connecting the syringe with the tissue to another syringe of the same size through a special connector.
9. Transferring the tissue from a syringe to the other one, vigorously 10 to 20 times in order to obtain a very fluid injectable tissue containing mesenchymal stem cells (ADSCs).

The most critical step of the above procedure is the harvesting, that is performed by moving back and forth the cannula just under the skin of the patient for collecting adipose tissue. This operation is performed by a certified plastic surgeon because an improper use of the cannula, which is relatively long, may cause wounds to deeper tissues or inner organs of the patient. Moreover, it is mandatory to harvest the adipose tissue in the very superficial layer above the skin: in this layer there is the highest concentration of SVF hence of ADSCs.

US 20110313345 discloses a device for removing adipose tissue from a surgical site or location in a patient's body. This prior device is designed to prevent an inexperienced operator from going too superficially or too deeply thanks to a “bar”, connected to a handpiece of the device, which guides the operator to the correct tissue harvesting, once placed in contact and tangentially to the patient's skin. The device includes a cannula which serves to provide infiltration, conduct ultrasonic energy device and also provide a conduit for aspiration to a fluid system used for infiltration and collection of fluids. An ultrasonic driver assembly is included into a handpiece of the device and is connected to an external fluid system. The external fluid system, installed outside of the handpiece of the device, comprises an external pump coupled to the cannula for aspiring a body fluid and for storing it into a reservoir installed outside the handpiece.

The connection from the cannula to the reservoir id established through a tubing, disposed mainly outside of the device, that hinders a proper handling of the device. Moreover, the device can be used only in connection with the ultrasonic driver assembly and with the external fluid system, which is equipped with the aspiring pump, thus it requires an electrical power source and it relatively cumbersome.

WO2014049713 discloses a syringe storage container configured to store a plurality of syringes that are each provided with a contacting part on a side face of a flange on an outer peripheral part of a cylindrical body part.

U.S. Pat. No. 4,713,053 discloses an apparatus for performing suction lipectomy having a guide surface adapted to contact and slide against the skin of a patient while the cannula tip is manually directed by the surgeon through the fatty tissue in reciprocating strokes. The guide is a separate piece fixed to a handpiece with bolts. Moreover, the cannula is coupled to an external suction means, which is installed outside of the handpiece and is connected to the cannula through an external tubing for aspiring a body fluid and for storing it into a reservoir installed outside the handpiece.

It would be desirable a safely device for guiding the insertion and the movement of the cannula below the skin of the patient, that would make impossible any incorrect and potentially dangerous use thereof, thus that could be handled also by a less qualified person than a certified plastic surgeon, and moreover guide the operator to harvest tissue in the best layer of adipose tissue.

### SUMMARY

An excellent solution to at least part of the above-mentioned drawbacks is provided by a guiding device as defined in claim 1. The guiding device of this disclosure is shaped so as to hold a syringe equipped with the harvesting cannula and to allow to insert this cannula at the exact depth below the skin of a patient to attain the subcutaneous adipose tissue. The cannula may be moved back and forth in a driven manner immersed in the subcutaneous adipose tissue without any risk of wounding inner organs of the patient. This disclosure provides also a syringe holder in the form of a hollow box with circular openings on a top surface. The circular openings are shaped so as to let a barrel of a syringe enter therein and the barrel flange not pass therethrough, in order to keep harvesting syringes hung with their barrel flanges abutted against the top surface of the syringe holder and their barrels protected into the hollow box.

The guiding device and the syringe holder may be marketed as a sterile kit for or taking adipose derived stem cells (ADSCs) from a subcutaneous adipose tissue below the skin of a patient.

The claims as filed are integral part of this specification and are herein incorporated by, reference.

### BRIEF DESCRIPTION OF THE DRAWINGS

FIG. 1 is a picture of three different harvesting cannulas.

FIG. 2 is a top view of a prototype of a guiding device according to this disclosure with a central locking tooth for retaining a syringe for collecting ADSCs through the harvesting procedure from the subcutaneous adipose tissue of a patient.

FIG. 3 is a side view of the guiding device of FIG. 2 holding a syringe for harvesting tissue inserted therein.

FIG. 4 is a detailed view of the front portion of the guiding device of FIG. 3 with the apical portion of the harvesting cannula protruding therefrom.

FIG. 5 schematically depicts how to use the guiding device of this disclosure for collecting ADSCs from subcutaneous adipose tissue without harming the patient.

FIGS. 6 and 7 are perspective views from different points of a guiding device according to another embodiment of this disclosure, with a side locking tooth for retaining the syringe.

FIG. 8 depicts an alternative embodiment of a guiding device of this disclosure, with two locking teeth for holding a flange of a syringe.

FIG. 9 is a picture of a prototype of a plunger lock for a syringe, having a tooth configured to engage with a corresponding side hole of the cylindrical holder to hinder a plunger of a syringe from advancing when the inside of the syringe is under vacuum.

FIGS. 10a, 10b and 10c show how to install the plunger lock onto the cylindrical holder of the guiding device to make the tooth engage with the side hole.

FIGS. 11a, 11b, 11c, 11d are different views of the plunger lock of FIG. 9 when mounted on the guiding device of this disclosure to stop the plunger of a syringe.

FIGS. 12 and 13 are a top view and a side view, respectively, of a prototype of a syringe holder of a kit of this disclosure for storing syringes filled with adipose tissue containing SVF and ADSCs.

FIG. 14 is a perspective view of the syringe holder of FIGS. 12 and 13.

FIGS. 15 to 25 and from 27 to 39 illustrate a sequence of operations to be performed for using the guiding device and the syringe holder of this disclosure.

FIG. 26 depicts a device of this disclosure for harvesting adipose tissue comprising the guiding device of FIG. 8 and a syringe with a harvesting cannula inserted in the hollow cylindrical holder.

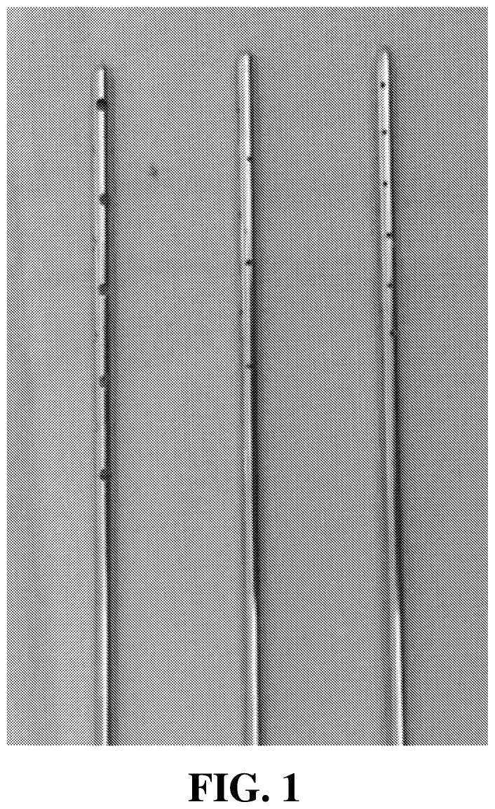

### DETAILED DESCRIPTION

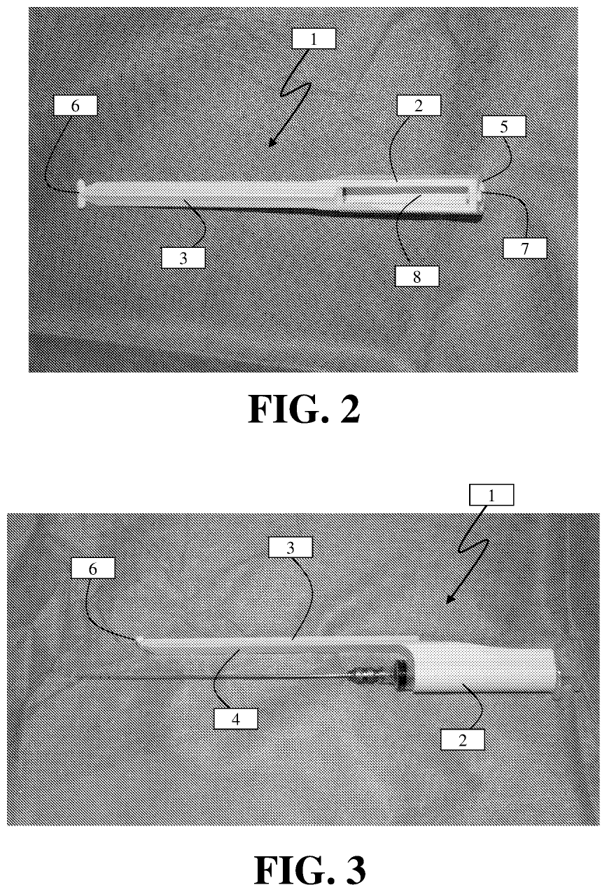

This disclosure provides a device, an embodiment of which is depicted in FIG. 2, for guiding insertion and movement of a harvesting cannula below the skin without any risk of harming the patient and at the correct depth in order to collect the adipose tissue with the highest concentration in SVF and ADSCs. The guiding device 1 substantially comprises a hollow cylindrical holder 2 of a syringe for harvesting procedure and a cantilever straight bar 3 integral with the cylindrical holder 2. As shown in FIG. 3, the cantilever straight bar 3 has a planar abutting surface 4 destined to come into contact with the skin of the patient.

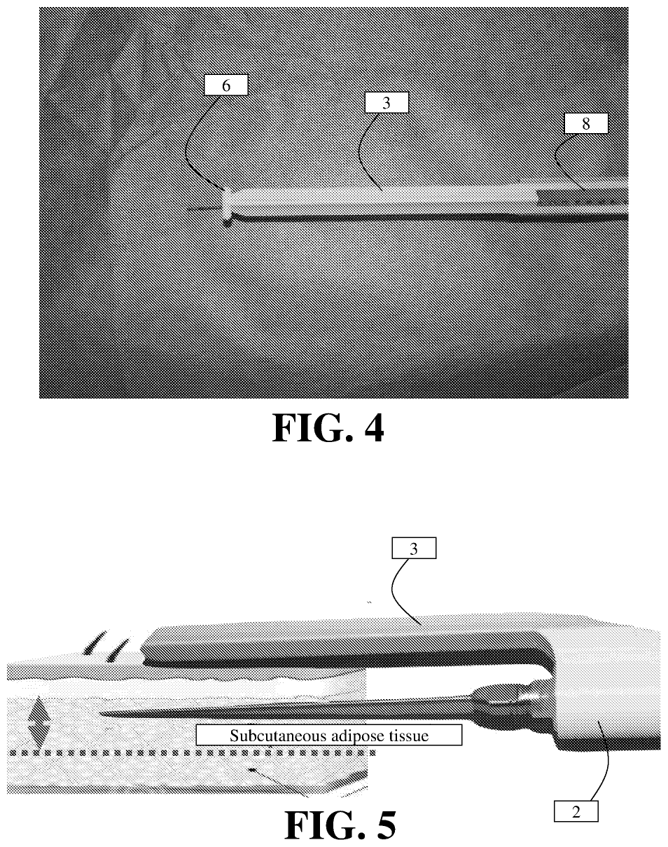

A syringe equipped with a harvesting cannula is inserted in the cylindrical holder 2, so as the barrel of the syringe is held coaxially with the holder 2 and the barrel flange is abutted against an end surface 5 of the holder. According to an aspect, the cylindrical holder 2 is shaped so as to keep the longitudinal axis of the harvesting cannula parallel to the planar abutting surface 4 and at a pre-established distance therefrom. The length of the cantilever straight bar 3 is determined in order to make the apical portion of the harvesting cannula protrude projectively beyond the tip 6 of the guiding device 1 exactly for said pre-established distance, as it may be better appreciated in FIGS. 4 and 5. This pre-established distance is determined so as to correspond to the depth at which the harvesting cannula should be inserted into the patient's skin to reach the subcutaneous adipose tissue.

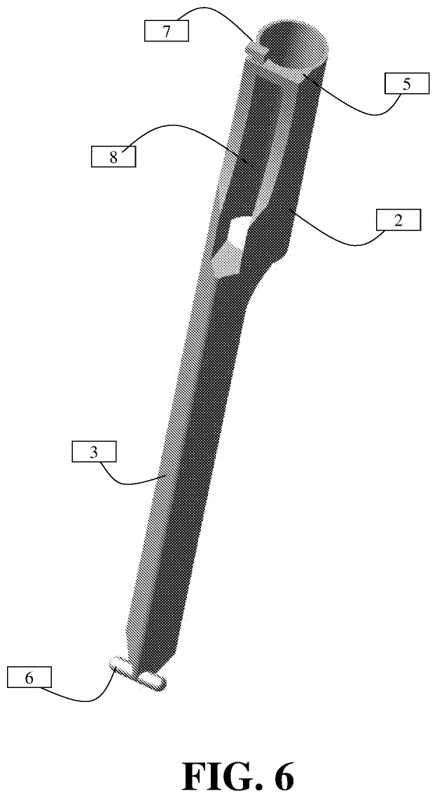

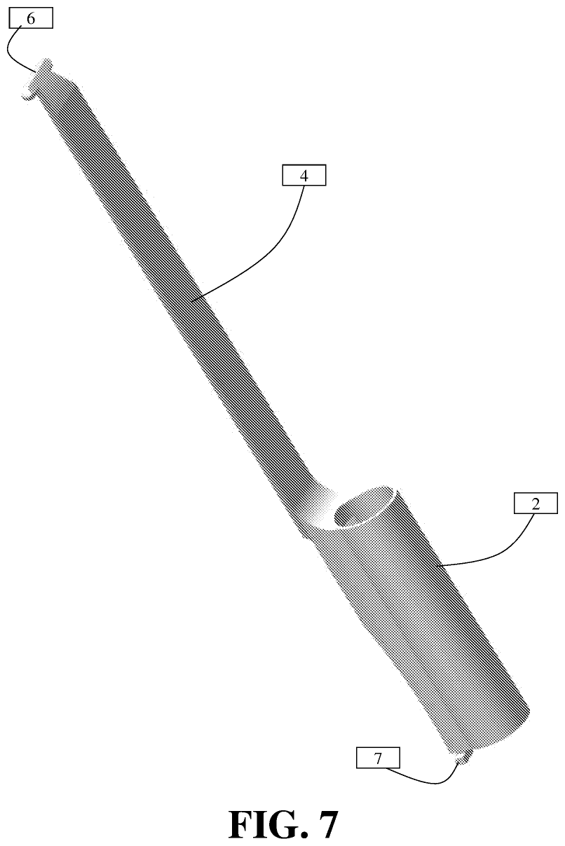

According to an aspect, the guiding device 1 has a locking tooth 7 integral with the cylindrical holder 2, that may be formed either in correspondence of a plane of symmetry of the cylindrical holder 2, as shown in FIG. 2, or at a distance therefrom, as shown in the other embodiment depicted in FIGS. 6 and 7. The locking tooth 7 is shaped so as to lock the barrel flange of the syringe, in order to make easier to pull the plunger backwards to create negative pressure inside the syringe. In practice, the syringe with the harvesting cannula is inserted in the cylindrical holder 2 and the barrel flange is rotated to as to engage the locking tooth 7 and being retained thereby. Clearly, the position of the locking tooth 7 is irrelevant provided that it allows to retain the barrel flange. In this configuration, an user may pull backwards the plunger of the syringe with the barrel firmly held by the guiding device 1.

FIG. 5 provides a pictorial explanation of how to use a guiding device of this disclosure. First, the end portion of the harvesting cannula is moved to penetrate throughout the skin as far as the tip 6 of the cantilever straight bar 3 comes into contact with the skin. When this happens, the cannula has attained the subcutaneous adipose tissue, and thus the syringe is rotated of 90 degrees so as to abut the planar surface 4 of the straight bar 3 against the abdomen skin. In this condition, the guiding device 1 is moved back and forth to collect subcutaneous adipose tissue whilst the planar abutting surface 4 of the cantilever straight bar 3 slides in contact with the skin of the abdomen of the patient.

With the guiding device 1 of this disclosure, the insertion of the cannula is driven exactly at the correct depth for being immersed in the subcutaneous adipose tissue, which may be for example an adipose layer of the abdomen, and its motion is guided so as to remain immersed therein without any risk of wounding inner organs of the patient and to harvest the tissue in the proper layer. It may be appreciated that insertion of the harvesting cannula and collection of adipose tissue is outstandingly simplified and is intrinsically safe, thus it may be carried out also by less qualified persons than the plastic surgeon.

According to an aspect, in order to prevent the patient from being scratched by sharp edges of the free end of the cantilever straight bar 3, a tip 6 with rounded edges is preferably formed integral with the cantilever straight bar 3. In the embodiments of FIGS. 4 and 7 the rounded tip 6 is substantially cylindrical, but any other shape free from sharp edges may be imparted.

According to an aspect, the hollow cylindrical holder 2 has a side opening 8 that leaves uncovered the barrel of the syringe, to make the user see how much adipose tissue is being aspired. According to an aspect, the side opening 8 is longitudinally aligned with the cantilever straight bar 3.

The guiding device may be made of plastic or of any material suitable to be used in contact with the patient's body during surgical operations.

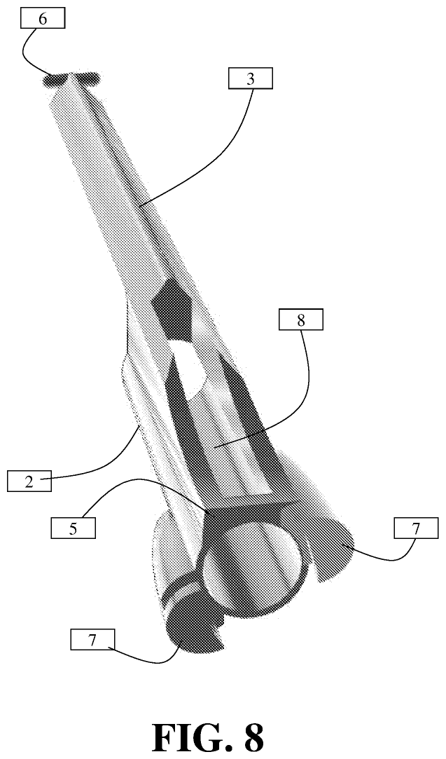

According to an alternative embodiment, depicted in FIG. 8, a guiding device of this disclosure may be equipped with two locking teeth 7 for locking a barrel flange of a syringe.

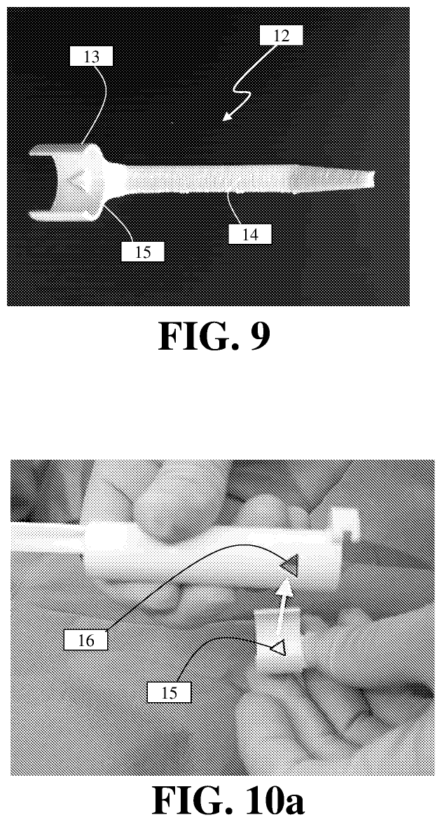

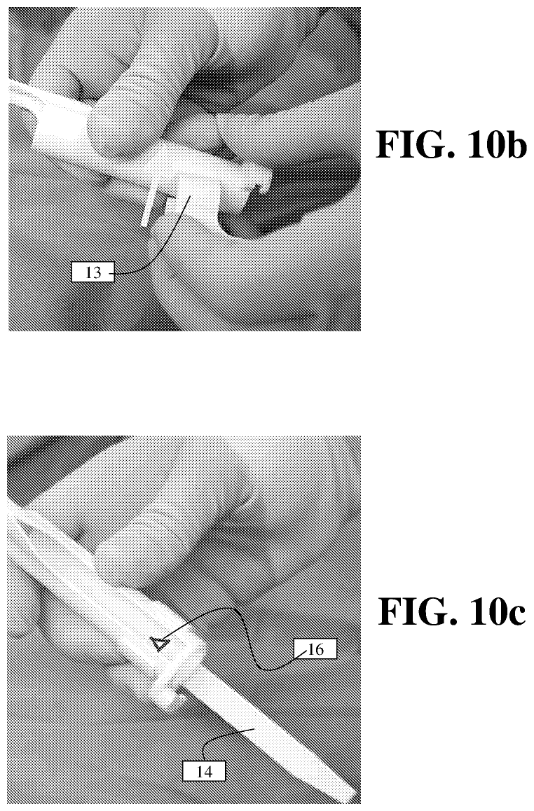

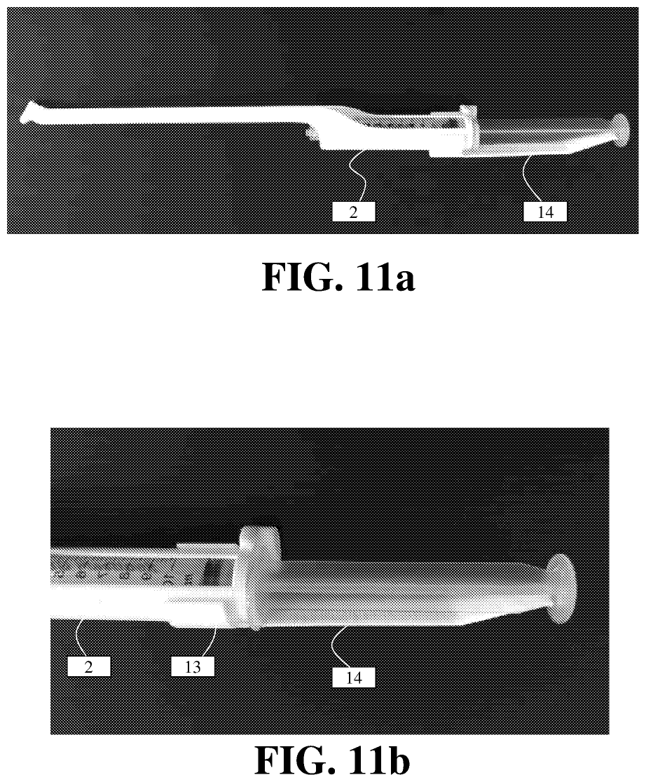

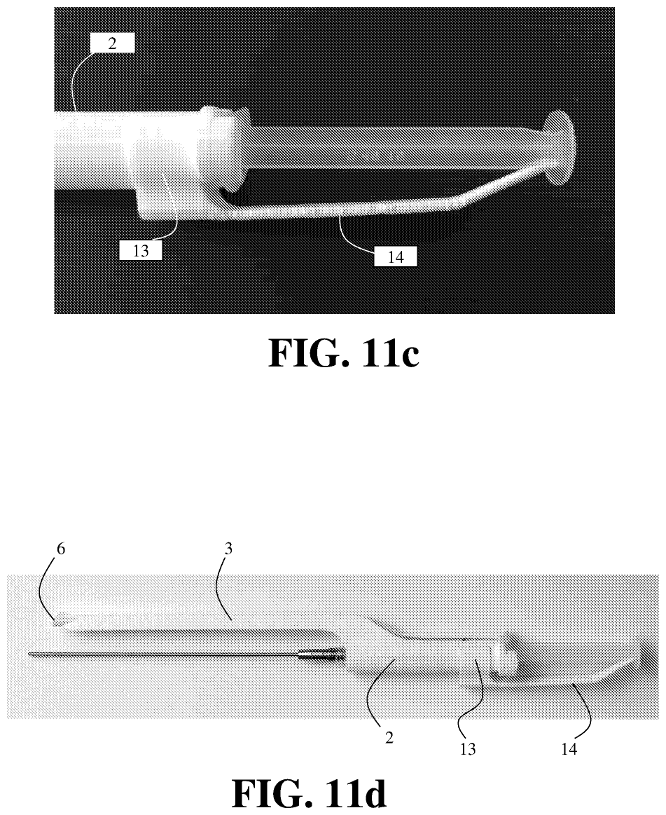

According to an aspect, the guiding device of this disclosure may be equipped with a plunger lock for a syringe of the type shown in FIG. 9. The plunger lock 12 has an elastically deformable transversal arched portion 13 configured to embrace at least partially the hollow cylindrical holder 2 so as to grasp it, and a longitudinal stem 14 integral with the arched portion 13. According to an aspect, on the internal surface of the arched portion 13 there may be a tooth 15 that protrudes inward. As shown in the sequence of pictures of FIGS. 10a to 10c, on the cylindrical holder 2 of the guiding device there is a hole or a recess 16 on the outer surface of the holder 2 that matches with the tooth 15 so as when the tooth 15 engages with the hole or recess 16 (FIG. 10c), the plunger lock 12 is hindered from sliding onto the outer surface of the holder 2. When the arched portion is mounted around the hollow cylindrical holder 2, as shown in the different views of FIGS. 11a to 11c, the stem 14 lays longitudinally and hinders the plunger of the syringe to advance when the syringe is under vacuum. Therefore, it is possible to keep the inside of the syringe under vacuum without using the fingers to keep the plunger in a retracted configuration.

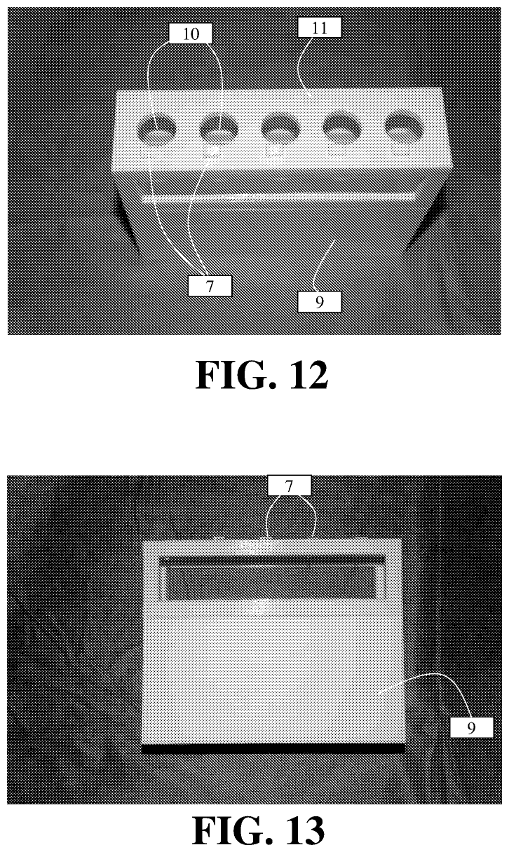

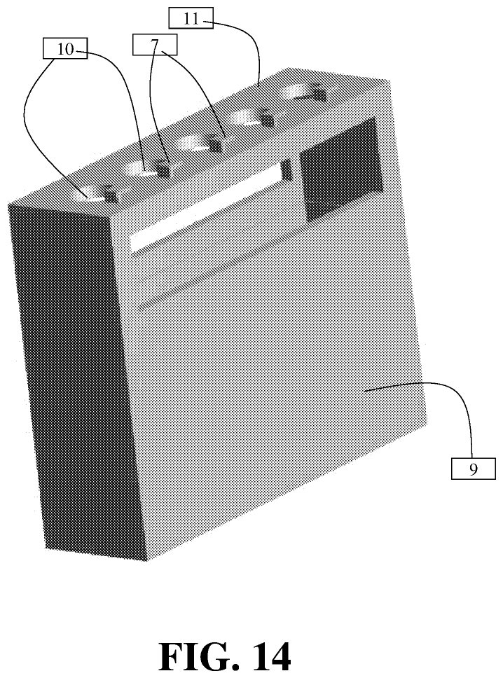

This disclosure provides also a syringe holder 9, of the type depicted in FIGS. 12 to 14. The syringe holder 9 may be realised as a hollow box with circular openings 10 on a top surface 11, wherein the circular openings 10 are shaped so as to let a barrel of a syringe enter therein and the barrel flange not pass therethrough. The syringes are hung with their barrel flanges abutted against the top surface 11 and the barrels protected into the hollow box pre-filled with cold Ringer lactate. Preferably, locking teeth 7 are realised on the top surface 11 and integral therewith, close to the circular openings 10 to lock the barrel flanges of syringes as in the cylindrical holder 2.

In practice, once the syringe is filled of subcutaneous adipose tissue up to about half the syringe capacity, the harvesting cannula is removed and the syringe is filled of Ringer lactate in order to washout the anaesthetic solution. Then a cover cap is put on the syringe adaptor and the syringe is placed at rest for a certain time in the hollow box of FIG. 8, with the syringe adaptor oriented toward the bottom. After a suitable amount of time, the tissue containing stem cells are separated and washing liquid to be discarded is accumulated in correspondence of the tip of the syringe, and thus it can be easily pushed out of the syringe.

The syringe holder 9 may be sold together with the guiding device 1 as a sterile kit for taking adipose derived stem cells from a subcutaneous adipose tissue below the skin of the patient.

The use of a guiding device and of a sterile kit of this disclosure is illustrated in the sequence of pictures from 15 to 25 and from 27 to 39.

The procedure is to be performed in sterile conditions: the skin should be carefully cleaned with a disinfecting solution both in the harvesting area and in the implantation area prior to the procedure.

The area where the tissue is harvested should be chosen based on the following criteria:
adequate quantity of subcutaneous adipose tissue;
size of the area: the harvesting area should be at least 20 cm in diameter;
the most common harvesting areas are: the abdomen, the hips and the trochanteric region.

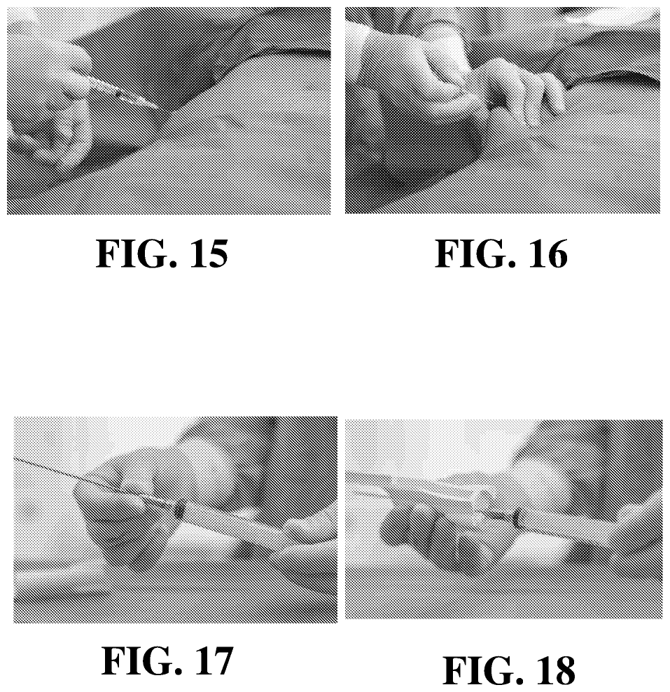

Using local anaesthetic containing adrenalin is recommended. Choose the spot for insertion of the cannula: it should be 1 cm out of the perimeter of the harvesting area (about 20 cm in diameter). Use the 27G needle to inject (FIG. 15) the anaesthetic solution.

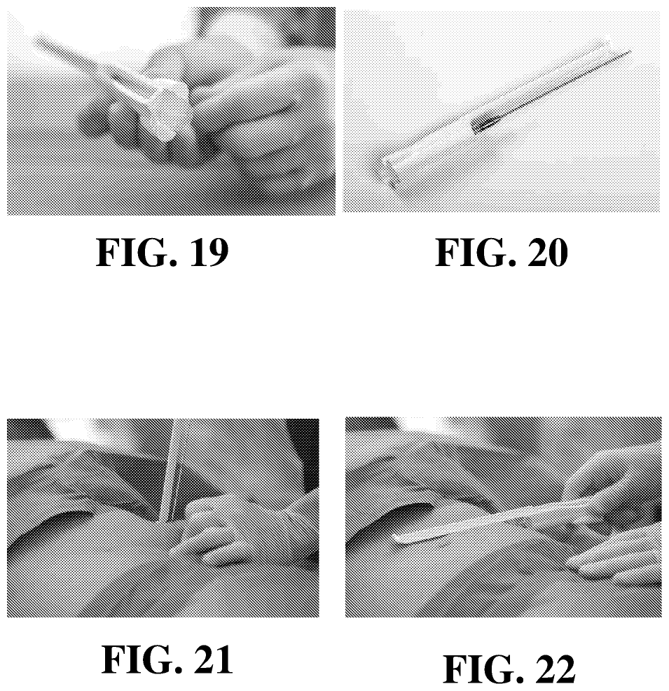

Make an opening in the skin (FIG. 16) with the 18G needle. Connect the multi-hole harvesting cannula with the luer lock syringe (FIG. 17) containing local anaesthetic. Insert the syringe with the cannula (FIG. 18) into the holder 2, then rotate the syringe (FIG. 19) so that the barrel flange is engaged in the locking teeth (or tooth) 7 defined on the base abutting surface 5 of the barrel flange. Make sure the cannula is parallel to the cantilever straight bar 3 (FIG. 20); if not so, set it in position with a delicate manual move.

Between thumb and forefinger of the non-dominant hand, pinch the skin (FIG. 21) where the opening has been previously made with the 18G needle and insert the tip of the cannula, then rotate the cannula by 90 degrees (FIG. 22) so as to set the external guide parallel to the skin plane.

Infiltrate the local anaesthesia with a back and forth movement of the cannula while maintaining the straight bar 3 parallel and with the planar abutting surface 4 adhering to the skin.

The anaesthetic solution should be injected in the whole harvesting area. If more anaesthetic is necessary, repeat the same procedure after recharging the syringe with more anaesthetic solution, and wait about 15 minutes.

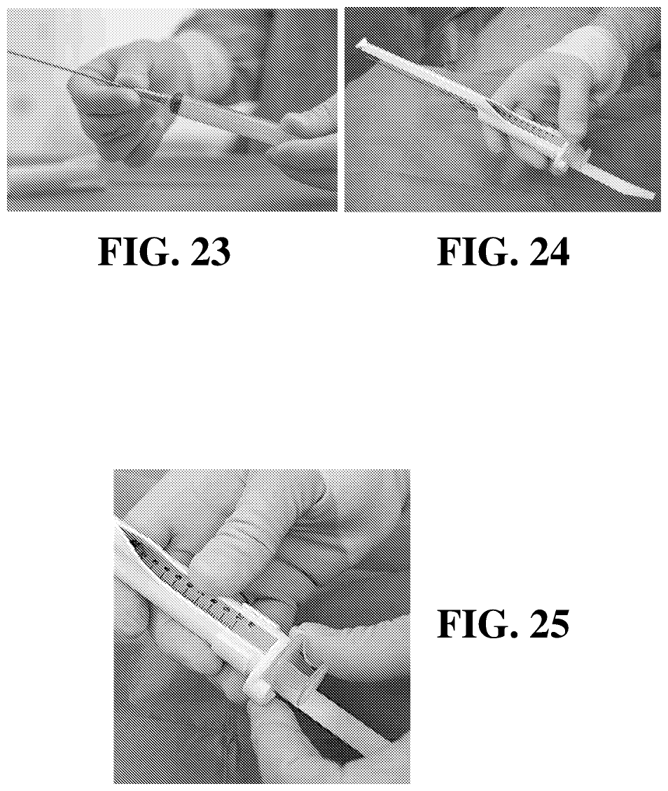

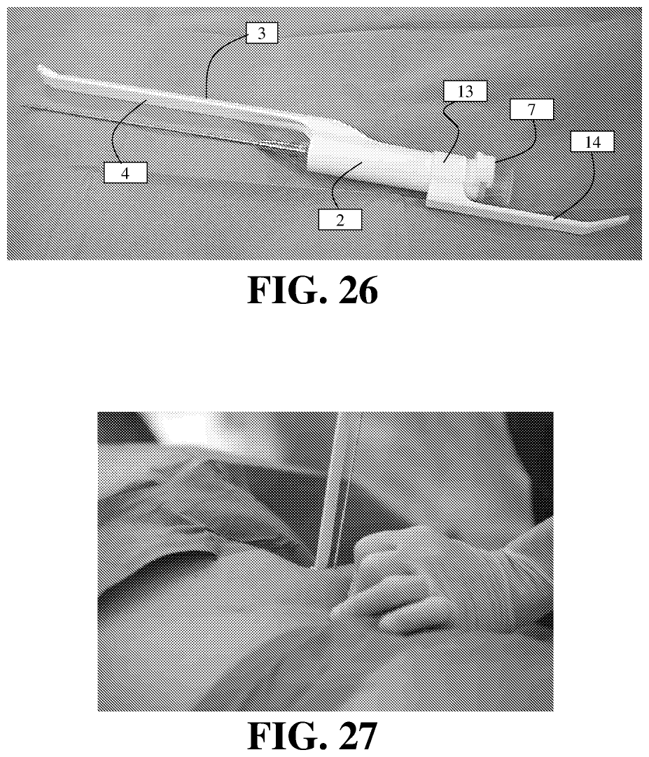

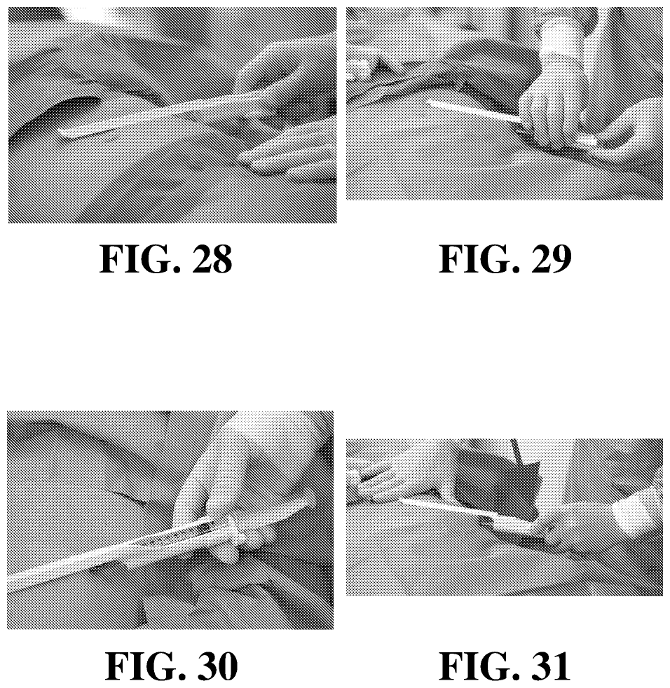

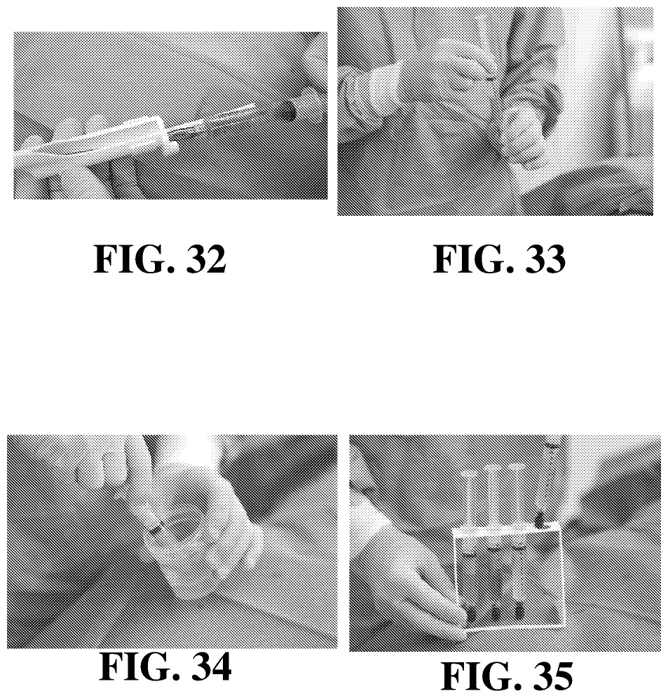

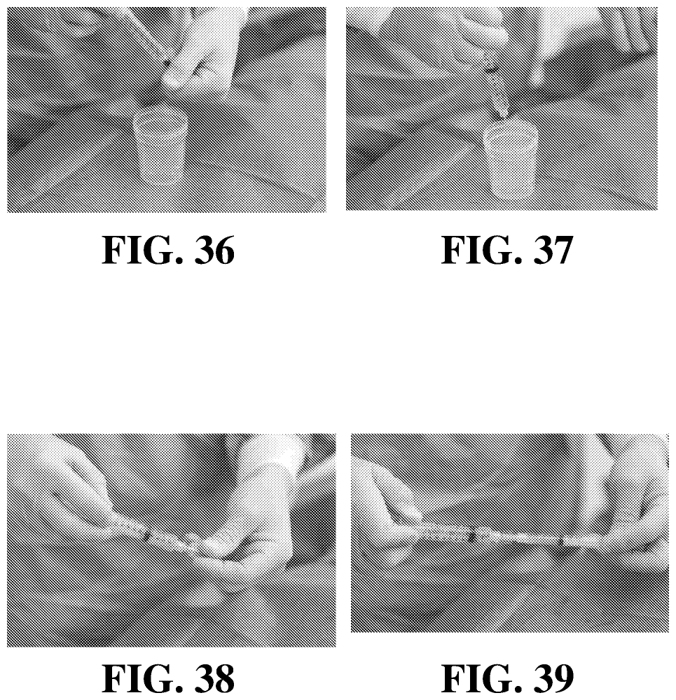

Connect the multi-hole harvesting cannula (FIG. 23) with the luer lock syringe, connect the plunger lock to the handpiece (FIGS. 10a, 10b), insert the syringe with the cannula (FIG. 24) into the cylindrical holder 2 and rotate the syringe (FIG. 25) so that the barrel flange is engaged in the locking teeth (or tooth) 7. Make sure the cannula is parallel to the cantilever straight bar 3; if not so, set it in position with a delicate manual move. The device for harvesting subcutaneous adipose tissue, comprising a guiding device and a syringe with a harvesting cannula inserted in the cylindrical holder 2, will appear as shown in FIGS. 11d and 26.

Between thumb and forefinger of the non-dominant hand, pinch the skin (FIG. 27) where the opening has been previously made with the 18G needle (FIG. 16), and insert the tip of the cannula into the skin opening perpendicularly until the tip 6 of the cantilever straight bar 3 touches the skin. Rotate the cannula by 90 degrees (FIG. 28) so as to set the cantilever straight bar 3 parallel to the skin plane and the cannula in a subcutaneous plane. While holding the device with the dominant hand (FIG. 29), pull the syringe plunger back so as to create the vacuum in the syringe barrel. The plunger lock system, of the type depicted in FIG. 9, will hold the plunger (FIG. 30) so as there is vacuum insider the barrel of the syringe without the need to maintain the plunger with the dominant hand.

While maintaining the cantilever straight bar 3 parallel and with the planar abutting surface adhering to the skin (FIG. 31), move the cannula back and forth in the whole harvesting area for sucking tissue into the barrel. The amount of harvested tissue can be checked through the side opening 8 in the hollow cylindrical holder 2, as schematically indicated by the arrow. Stop as soon as about half the syringe capacity of tissue has been collected in the syringe.

Pull out the cannula from the skin and rotate the syringe until the plunger flange is released from the locking teeth (or tooth) 7 in the back of the holder 2. Pull out the syringe (FIG. 32) from the guide and disconnect the cannula (FIG. 33). Fill the syringe with sterile Ringer Lactate (FIG. 34) previously poured into a container and use a luer lock plug to close the syringe. Put the syringe (plug down) in the syringe holder 9 of the sterile kit (FIG. 35). After few minutes, the syringes in the syringe holder 9 will show a separation by gravity of the tissue (top) from the Ringer lactate solution (bottom).

Pull out the syringe from the syringe holder 9, hold it in a vertical position without tilting it or turning it upside down, remove the plug (FIG. 36) and discard the washing Ringer Lactate solution (FIG. 37) while retaining all the tissue inside the syringe. At the end of this procedure a fat tissue with SVF and ADSCs is obtained; this tissue is already enough fluid to be injected.

According with the Regenerative therapy purposes, the tissue could be concentrated in term of cellularity through a gently centrifugation, or increase the fluidity despite of the viability of adipocyte through a procedure of tissue transfer between to syringes. In any case it is suggested to transfer the tissue in smaller syringes.

Pull out the syringe from the stand, remove the plug (FIG. 38) and connect the luer lock transfer connector. Connect the syringe at the other end of the transfer connector (FIG. 39) and transfer the tissue, which is now ready to be injected.

1. A guiding device for inserting and guiding a harvesting cannula below the skin of a patient, comprising:
a handpiece defining a hollow cylindrical holder configured to hold in a removable fashion a barrel of a syringe equipped with a harvesting cannula;
a cantilever straight bar integral with the cylindrical holder having a planar abutting surface adapted to slide in contact with the skin of the patient whilst a harvesting cannula of a syringe housed into said hollow cylindrical holder is immersed in a subcutaneous adipose tissue below the skin;
wherein said hollow cylindrical holder, is shaped so as to hold a longitudinal axis of a harvesting cannula, of a syringe housed into said hollow cylindrical holder, parallel to said planar abutting surface and at a first distance from said planar abutting surface equal to a depth at which there is said subcutaneous adipose tissue below the skin;
wherein said cantilever straight bar has a longitudinal length determined so as to make a tip of a harvesting cannula, of a syringe housed into said hollow cylindrical holder, protrude projectively beyond a free end of the cantilever straight bar exactly for said first distance;
wherein said hollow cylindrical holder has a side opening configured to let a user to see a barrel of a syringe housed into said hollow cylindrical holder whilst it is filled with cells of said subcutaneous adipose tissue.

2. The guiding device of claim 1, wherein said cantilever straight bar terminates with a rounded tip in correspondence of said free end.

3. The guiding device of claim 1, wherein said side opening is longitudinally aligned with said cantilever straight bar.

4. The guiding device of claim 1, wherein said hollow cylindrical holder has a base abutting surface for a barrel flange of a syringe, and a locking tooth integral with the hollow cylindrical holder for retaining a barrel flange of a syringe housed into the hollow cylindrical holder whilst a plunger of the syringe is pulled backwards.

5. The guiding device of claim 1, further comprising a plunger lock for a syringe, said plunger lock having:
an elastically deformable transversal arched portion configured to embrace at least partially said hollow cylindrical holder, and
a longitudinal stem integral with said arched portion, configured to hinder a plunger of a syringe, housed into the hollow cylindrical holder, to move towards the inside of the syringe when said arched portion is tightly fit around said hollow cylindrical holder.

6. A device for harvesting subcutaneous adipose tissue, comprising:
a guiding device as defined in claim 1;
a syringe having a barrel, a plunger and a harvesting cannula functionally fixed to said barrel, wherein the barrel is removably housed into said hollow cylindrical holder.

7. A sterile kit for harvesting adipose derived stem cells from a subcutaneous adipose tissue below the skin of a patient, comprising:
a guiding device according to claim 1, and
a syringe holder in the form of a hollow box with circular openings on a top surface of the hollow box, said circular openings being shaped so as to let a barrel of a syringe enter therein and a barrel flange not pass therethrough, configured to keep harvesting syringes hung with their barrel flanges abutted against said top surface and their barrels protected into said hollow box.

8. The sterile kit of claim 7, wherein said syringe holder comprises locking teeth for retaining barrel flanges, each locking tooth of said locking teeth being integral with said top surface and being formed at a perimeter of a corresponding circular opening of said circular openings.

9. The guiding device of claim 2, wherein said side opening is longitudinally aligned with said cantilever straight bar.

10. The guiding device of claim 9, wherein said hollow cylindrical holder has a base abutting surface for a barrel flange of a syringe, and a locking tooth integral with the hollow cylindrical holder for retaining a barrel flange of a syringe housed into the hollow cylindrical holder whilst a plunger of the syringe is pulled backwards.

11. The guiding device of claim 10, further comprising a plunger lock for a syringe, said plunger lock having:
an elastically deformable transversal arched portion configured to embrace at least partially said hollow cylindrical holder, and
a longitudinal stem integral with said arched portion, configured to hinder a plunger of a syringe, housed into the hollow cylindrical holder, to move towards the inside of the syringe when said arched portion is tightly fit around said hollow cylindrical holder.
